(19) United States
(12) Patent Application Publication
(10) Pub. No.: US 2007/0073212 A1
(43) Pub. Date: Mar. 29, 2007

(54) IONTOPHORESIS APPARATUS AND METHOD TO DELIVER ACTIVE AGENTS TO BIOLOGICAL INTERFACES

(76) Inventor: Takehiko Matsumura, Shibuya-ku (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 11/532,049

(22) Filed: Sep. 14, 2006

Related U.S. Application Data
(60) Provisional application No. 60/721,843, filed on Sep. 28, 2005.

(51) Int. Cl. A61N 1/30

(52) U.S. Cl. 604/20

(57) ABSTRACT

An iontophoresis device includes active and counter electrode assemblies. The active electrode assembly includes an active electrode element, an outermost ion selective membrane caching an active agent and a further active agent carried by an outer surface of the outermost ion selective membrane. The active electrode assembly may also include an inner active agent reservoir storing additional active agent, an electrolyte reservoir storing electrolyte, an inner ion selective membrane positioned between the electrolyte reservoir and the active agents. The active electrode may also include an inner withdrawable sealing liner between the electrolyte reservoir and the active agents. An outer release liner may protectively cover or overlay the further active agent and/or outer surface prior to use.

### CROSS-REFERENCE TO RELATED APPLICATION

This application claims the benefit under 35 U.S.C. § 119(e) of U.S. Provisional Patent Application No. 60/721,843, filed Sep. 28, 2005.

### BACKGROUND OF THE INVENTION

1. Field of the Invention

This disclosure generally relates to the field of iontophoresis, and more particularly to the effective delivery of active agents such as therapeutic agents or drugs to a biological interface under the influence of electromotive force.

2. Description of the Related Art

Iontophoresis employs an electromotive force to transfer an active agent such as an ionic drug or other therapeutic agent to a biological interface such as skin or mucus membrane.

Iontophoresis devices typically include an active electrode assembly and a counter electrode assembly, each coupled to opposite poles or terminals of a voltage source, such as a chemical battery. Each electrode assembly typically includes a respective electrode element to apply an electromotive force. Such electrode elements often comprise a sacrificial element or compound, for example silver or silver chloride.

The active agent may be either cation or anion, and the voltage source can be configured to apply the appropriate voltage polarity based on the polarity of the active agent. Iontophoresis may be advantageously used to enhance or control the delivery rate of the active agent. The active agent may be stored in a reservoir such as a cavity, or stored in a porous structure or as a gel. As discussed in U.S. Pat. No. 5,395,310, an ion exchange membrane may be positioned to serve as a polarity selective barrier between the active agent reservoir and the biological interface.

An ion exchange membrane may comprise large pores in order to compensate for active agents with large molecular weights. The pores may be larger than the active agent being administered. Large pores reduce the capacity of the ion exchange membrane to be ion selective, thereby decreasing the active agent delivery rate of the iontophoresis device.

Ion exchange membranes may bind ions having a high transport number, which are not active agents such as, for example, Na+, H+, or Cl−. Such bonded ions may replace the active agents and be advantageously delivered into the biological interface instead of the active agent, thereby hampering the active agent delivery rate.

Positioning the ion exchange membrane between the active agent reservoir and the biological interface may result in partial contact. This may allow counter ions to flow out of the biological interface and further reduce the active agent delivery rate.

Commercial acceptance of iontophoresis devices is dependent on a variety of factors, such as cost to manufacture, shelf-life or stability during storage, efficiency of active agent delivery, safety of operation, and disposal issues. An iontophoresis device that addresses one or more of these factors is desirable.

### BRIEF SUMMARY OF THE INVENTION

An iontophoresis device includes an active electrode element operable to provide an electrical potential, an outermost ion selective membrane having an outer surface, and a first amount of active agent carried by at least a portion of the outer surface of the outermost ion selective membrane prior to use in the absence of an electromotive force or current. An additional second amount of active agent may be cached within the outermost ion selective membrane.

In another embodiment of the invention, an iontophoresis device includes an active electrode element operable to provide an electrical potential, an outermost ion selective membrane having an outer surface, a first amount of active agent adhered to at least a portion of the outer surface of the outermost ion selective membrane, and an outer release liner covering the first amount of active agent and at least a portion of the outer surface of the outermost selective membrane prior to use. An additional second amount of active agent may be cached within the outermost ion selective membrane.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn are not intended to convey any information regarding the actual shape of the particular elements and have been solely selected for ease of recognition in the drawings.

FIG. 1 is a block diagram of an iontophoresis device comprising active and counter electrode assemblies according to one illustrated embodiment where the active electrode assembly includes an outermost membrane caching an active agent, active agent adhered to an outer surface of the outermost membrane and a removable outer release liner overlying or covering the active agent and outermost membrane.

FIG. 2 is a block diagram of the iontophoresis device of FIG. 1 positioned on a biological interface, with the outer release liner removed to expose the active agent according to one illustrated embodiment.

FIG. 3A is a schematic diagram of a portion of the iontophoresis device, showing a removable liner fully positioned between an inner ion exchange membrane and the active agent caching outermost membrane to prevent migration of the active agent during storage, according to one illustrated embodiment.

FIG. 3B is a schematic diagram of a portion of the iontophoresis device, showing the removable liner partially withdrawn from the position between the inner ion exchange membrane and the active agent caching outermost membrane in preparation for use, according to one illustrated embodiment.

FIG. 3C is a schematic diagram of a portion of the iontophoresis device, showing the removable liner fully withdrawn from the position between the inner ion exchange membrane and the active agent caching outermost membrane to allow transfer of ions between the various portions of the device during use, according to one illustrated embodiment.

### DETAILED DESCRIPTION OF THE INVENTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with iontophoresis devices, for example controllers including but not limited to voltage regulators, have not been shown or described in detail so as not to obscure the embodiments of the present invention.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein and in the claims, the term “membrane” means a layer, barrier or material, which may or may not be permeable. Unless specified otherwise, membranes may take the form of a solid, liquid or gel, and may or may not have a distinct lattice or cross-linked structure.

As used herein and in the claims, the term “ion selective membrane” means a membrane that is substantially selective to ions, passing certain ions while blocking passage of other ions. An ion selective membrane may, for example, take the form of a charge selective membrane, or may take the form of a semi-permeable membrane.

As used herein and in the claims, the term “charge selective membrane” means a membrane which substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. Examples of commercially available cation exchange membranes include those available under the designators NEOSEPTA, CM-1, CM-2, CMX, CMS, and CMB from Tokuyama Co., Ltd. Examples of commercially available anion exchange membranes include those available under the designators NEOSEPTA, AM-1, AM-3, AMX, AHA, ACH and ACS also from Tokuyama Co., Ltd.

As used herein and in the claims, the term bipolar membrane means a membrane that is selective. Unless specified otherwise, a bipolar membrane may take the form of a unitary membrane structure or multiple membrane structure. The unitary membrane structure may have a first portion including cation ion exchange material or groups and a second portion opposed to the first portion, including anion ion exchange material or groups. The multiple membrane structure may be formed by a cation exchange membrane attached or coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that substantially selective based on a size or molecular weight of the ion. Thus, a semi-permeable membrane substantially passes ions of a first molecular weight or size, while substantially blocking passage of ions of a second molecular weight or size, greater than the first molecular weight or size.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

A used herein and in the claims, the term “reservoir” means any form of mechanism to retain an element or compound in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound.

The headings provided herein are for convenience only and do not interpret the scope or meaning of the embodiments.

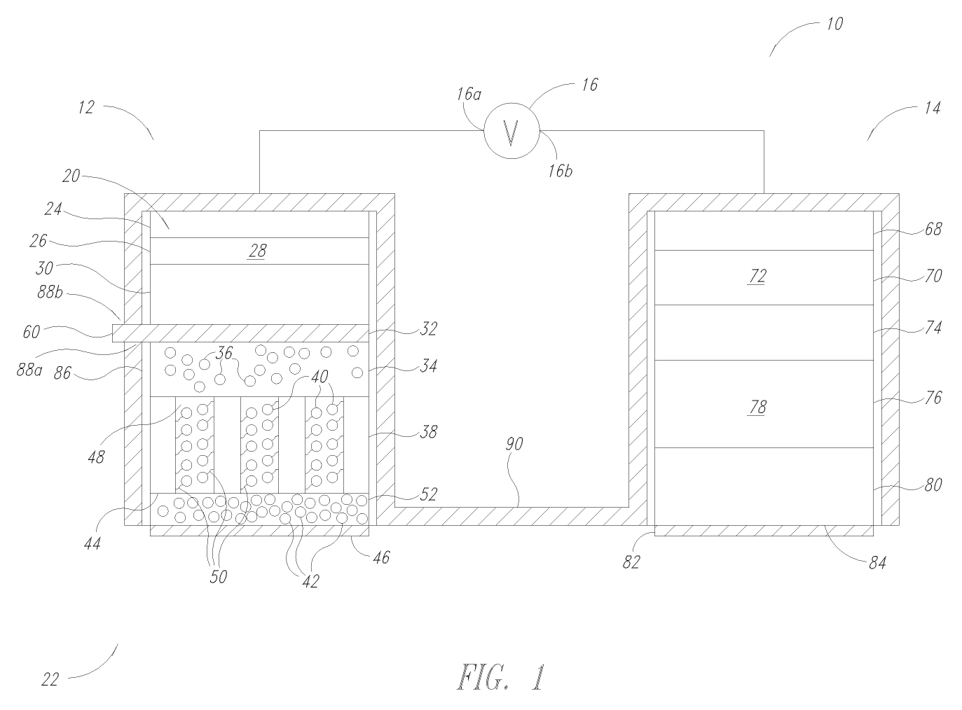

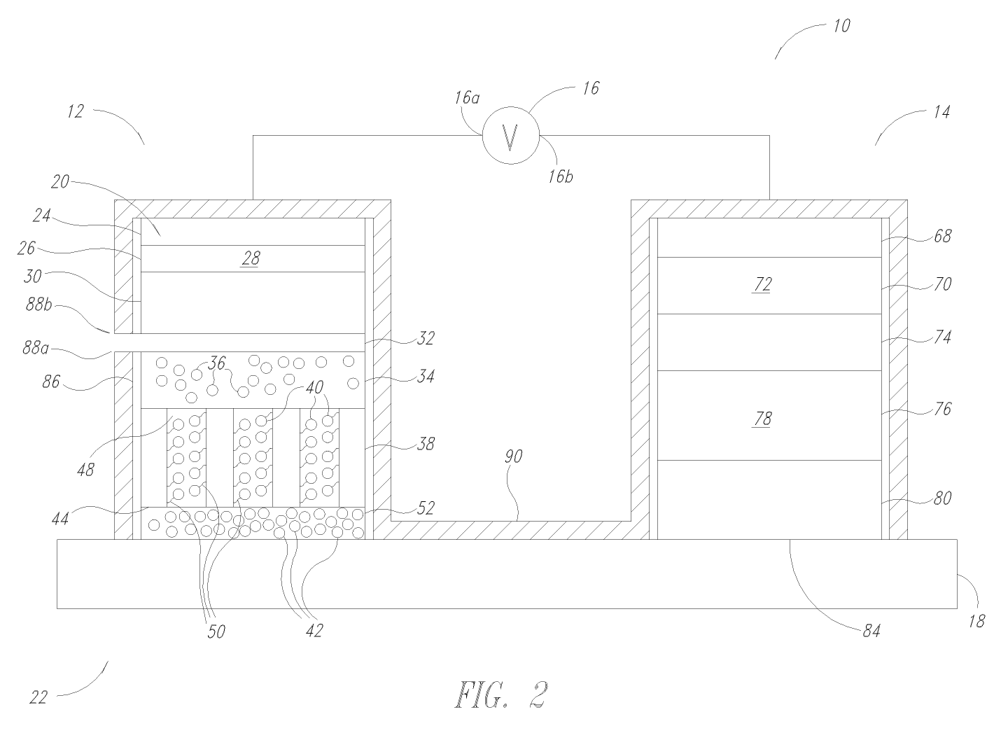

FIGS. 1 and 2 show an iontophoresis device 10 comprising active and counter electrode assemblies, 12, 14, respectively, electrically coupled to a voltage source 16, operable to supply an active agent to a biological interface 18a, 18b, such as a portion of skin or mucous membrane via iontophoresis, according to one illustrated embodiment.

In the illustrated embodiment, the active electrode assembly 12 comprises, from an interior 20 to an exterior 22 of the active electrode assembly 12, an active electrode element 24, an electrolyte reservoir 26 storing an electrolyte 28, an inner ion selective membrane 30, an inner sealing liner 32, an inner active agent reservoir 34 storing active agent 36, an outermost ion selective membrane 38 that caches additional active agent 40, further active agent 42 carried by an outer surface 44 of the outermost ion selective membrane 38, and an outer release liner 46. Each of the above elements or structures will be discussed in detail below.

The active electrode element 24 is coupled to a first pole 16a of the voltage source 16 and positioned in the active electrode assembly 12 to apply an electromotive force or current to transport active agent 36, 40, 42 via various other components of the active electrode assembly 12. The active electrode element 24 may take a variety of forms. For example, the active electrode element 24 may include a sacrificial element, for example a chemical compound or amalgam including silver (Ag) or silver chloride (AgCl). Such compounds or amalgams typically employ one or more heavy metals, for example lead (Pb), which may present issues with regard manufacturing, storage, use and/or disposal. Consequently, some embodiments may advantageously employ a carbon-based active electrode element 24. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004.

The electrolyte reservoir 26 may take a variety of forms including any structure capable of retaining electrolyte 28, and in some embodiments may even be the electrolyte 28 itself, for example, where the electrolyte 28 is in a gel, semi-solid or solid form. For example, the electrolyte reservoir 26 may take the form of a pouch or other receptacle, a membrane with pores, cavities or interstices, particularly where the electrolyte 28 is a liquid.

The electrolyte 28 may provide ions or donate charges to prevent or inhibit the formation of gas bubbles (e.g., hydrogen) on the active electrode element 24 in order to enhance efficiency and/or increase delivery rates. This elimination or reduction in electrolysis may in turn inhibit or reduce the formation of acids and/or bases (e.g., H⁺ ions, OH⁻ ions), that would otherwise present possible disadvantages such as reduced efficiency, reduced transfer rate, and/or possible irritation of the biological interface 18. As discussed further below, in some embodiments the electrolyte 28 may provide or donate ions to substitute for the active agent. for example substituting for the active agent 40 cached thereon. Such may facilitate transfer of the active agent 40 to the biological interface 18, for example, increasing and/or stabilizing delivery rates. A suitable electrolyte may take the form of a solution of 0.5 M disodium fumarate: 0.5 M Poly acrylic acid (5:1).

The inner ion selective membrane 30 is generally positioned to separate the electrolyte 28 and the inner active agent reservoir 34. The inner ion selective membrane 30 may take the form of a charge selective membrane. For example, where the active agent 36, 40, 42 comprises a cationic active agent, the inner ion selective membrane 38 may take the form of an anion exchange membrane, selective to substantially pass anions and substantially block cations. Also, for example, where the active agent 36, 40, 42 comprises an anionic active agent, the inner ion selective membrane 38 may take the form of an cationic exchange membrane, selective to substantially pass cations and substantially block anions. The inner ion selective membrane 38 may advantageously prevent transfer of undesirable elements or compounds between the electrolyte 28 and the active agents 26, 40, 42. For example, the inner ion selective membrane 38 may prevent or inhibit the transfer of hydrogen (H⁺) or sodium (Na⁺) ions from the electrolyte 72, which may increase the transfer rate and/or biological compatibility of the iontophoresis device 10.

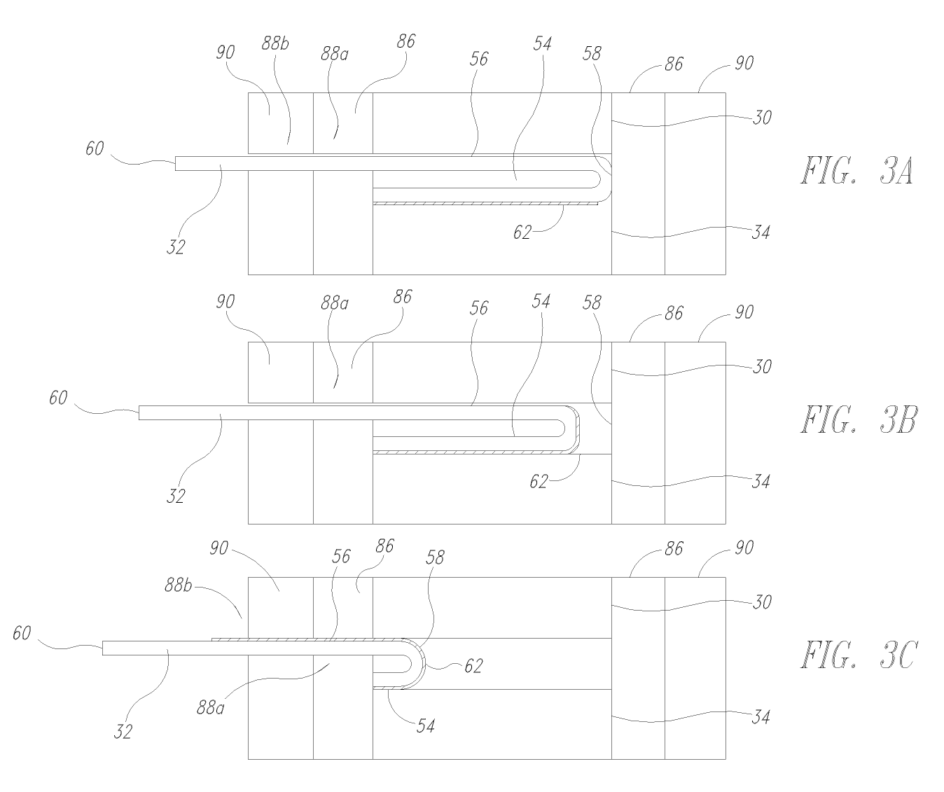

The inner sealing liner 32 separates the active agent 36, 40, 42 from the electrolyte 28 and is selectively removable, as discussed in detail below with respect to FIGS. 3A-3B. The inner sealing liner 32 may advantageously prevent migration or diffusion between the active agent 36, 40, 42 and the electrolyte 28, for example, during storage.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36, and in some embodiments may even be the active agent 36 itself, for example, where the active agent 36 is in a gel, semi-solid or solid form. For example, the inner active agent reservoir 34 may take the form of a pouch or other receptacle, a membrane with pores, cavities or interstices, particularly where the active agent 36 is a liquid. The inner active agent reservoir 34 may advantageously allow larger doses of the active agent 36 to be loaded in the active electrode assembly 12.

The outermost ion selective membrane 38 is positioned generally opposed across the active electrode assembly 12 from the active electrode element 24. The outermost membrane 38 may, as in the embodiment illustrated in FIGS. 1 and 2, take the form of an ion exchange membrane, pores 48 (only one called out in FIGS. 1 and 2 for sake of clarity of illustration) of the ion selective membrane 38 including ion exchange material or groups 50 (only three called out in FIGS. 1 and 2 for sake of clarity of illustration). Under the influence of an electromotive force or current, the ion exchange material or groups 50 selectively substantially passes ions of the same polarity as active agent 36, 40, while substantially blocking ions of the opposite polarity. Thus, the outermost ion exchange membrane 38 is charge selective. Where the active agent 36, 40, 42 is a cation (e.g., lidocaine), the outermost ion selective membrane 38 may take the form of a cation exchange membrane. Alternatively, where the active agent 36, 40, 42 is an anion, the outermost ion selective membrane 38 may take the form of an anion exchange membrane.

The outermost ion selective membrane 38 may advantageously cache active agent 40. In particular, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of semi-permeable or microporous membrane which is selective by size. In some embodiments, such a semi-permeable membrane may advantageously cache active agent 40, for example by employing the removably releasable outer release liner 46 to retain the active agent 40 until the outer release liner 46 is removed prior to use.

The outermost ion selective membrane 38 may be preloaded with the additional active agent 40, such as ionized or ionizable drugs or therapeutic agents. Where the outermost ion selective membrane 38 is an ion exchange membrane, a substantial amount of active agent 40 may bond to ion exchange groups 50 in the pores, cavities or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness or coverage as to constitute a layer in a conventional sense of such term.

The active agent 42 may be deposited in a variety of highly concentrated forms such as, for example, solid form, nearly saturated solution form or gel form. If in solid form, a source of hydration may be provided, either integrated into the active electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38. In such an embodiment, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 3, 40, 42 will all be of common polarity to prevent the active agents 36, 40, 42 from competing with one another. Other combinations are possible.

The outer release liner 46 may generally be positioned overlying or covering further active agent 42 carried by the outer surface 44 of the outermost ion selective membrane 38. The outer release liner 46 may protect the further active agent 42 and/or outermost ion selective membrane 38 during storage, prior to application of an electromotive force or current. The outer release liner 46 may be a selectively releasable liner made of waterproof material, such as release liners commonly associated with pressure sensitive adhesives. Note that the inner release liner 46 is shown in place in FIG. 1 and removed in FIG. 2.

An interface coupling medium (not shown) may be employed between the electrode assembly and the biological interface 18. The interface coupling medium may, for example, take the form of an adhesive and/or gel. The gel may, for example, take the form of a hydrating gel.

FIG. 3A shows the inner sealing liner 32 having a pair of legs 54, 56 and meniscus or elbow 58 formed therebetween. An end of one of the legs 56 forms a tab 60. The tab 60 extends from the active electrode assembly 12 to an exterior 62 thereof to allow selective withdrawal of the inner sealing liner 32 by a user just prior to use. As discussed above, the inner sealing liner 32 separates the active agent 36, 40, 42 from the electrolyte 28, to prevent migration or diffusion, particularly during storage. As illustrated in FIGS. 3B and 3C, the inner sealing liner 32 is removable by pulling on the tab 60. For example, FIG. 3B shows the inner sealing liner 32 partially withdrawn, while FIG. 3C shows the inner sealing liner 32 fully withdrawn to expose the active agent 36, 40, 42 to the electrolyte 28. The meniscus or elbow 58 travels along the inner sealing liner 32, changing the relative lengths of the legs 54, 56 as the tab 60 is pulled.

The side of the inner sealing liner 38 facing the inner active agent reservoir 34 may include a release agent 62, particularly where the active agent reservoir 34 is made from a material having adhesive characteristics. Otherwise, a thin waterproof material may suffice.

The counter electrode assembly 14 allows completion of an electrical path between poles 16a, 16b of the voltage source 16 via the active electrode assembly 12 and the biological interface 18. The counter electrode assembly 14 may take a variety of forms suitable for closing the circuit by providing a return path.

In the embodiment illustrated in FIGS. 1 and 2, the counter electrode assembly comprises, in order to form an interior 64 to an exterior 66 of the counter electrode assembly 14: a counter electrode element 68, electrolyte reservoir 70 storing an electrolyte 72, an inner ion selective membrane 74, an optional buffer reservoir 76 storing buffer material 78, an outermost ion selective membrane 80, and an outer release liner 82.

The counter electrode element 68 is electrically coupled to a second pole 16b of the voltage source 16, the second pole 16b having an opposite polarity to the first pole 16a. The counter electrode element 68 may take a variety of forms. For example, the counter electrode element 68 may include a sacrificial element, such as a chemical compound or amalgam including silver (Ag) or silver chloride (AgCl), or may include a non-sacrificial element such as the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities or interstices, particularly where the electrolyte 72 is a liquid.

The electrolyte 72 is generally positioned between the counter electrode element 68 and the outermost ion selective membrane 80, proximate the counter electrode element 68. The electrolyte 72 may provide ions or donate charges to prevent or inhibit the formation of gas bubbles (e.g., hydrogen) on the counter electrode element 68 and may prevent or inhibit the formation of acids or bases or neutralize the same, which may enhance efficiency and/or reduce the potential for irritation of the biological interface 18.

The inner ion selective membrane 74 is positioned between and/or to separate, the electrolyte 72 from the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydrogen (H⁺) or sodium (Na⁺) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane or a gel.

The buffer material 78 may supply ions for transfer through the outermost ion selective membrane 80 to the biological interface 18. Consequently, the buffer material 78 may, for example, comprise a salt (e.g., NaCl).

The outermost ion selective membrane 80 of the counter electrode assembly 14 may take a variety of forms. For example, the outermost ion selective membrane 80 may take the form of a charge selective ion exchange membrane, such as a cation exchange membrane or an anion exchange membrane, which substantially passes and/or blocks ions based on the charge carried by the ion. Examples of suitable ion exchange membranes are discussed above. Alternatively, the outermost ion selective membrane 80 may take the form of a semi-permeable membrane that substantially passes and/or blocks ions based on size or molecular weight of the ion.

The outermost ion selective membrane 80 of the counter electrode assembly 14 is selective to ions with a charge or polarity opposite to that of the outermost ion selective membrane 38 of the active electrode assembly 12. Thus, for example, where the outermost ion selective membrane 38 of the active electrode assembly 12 allows passage of negatively charged ions of the active agent 36,40, 42 to the biological interface 18, the outermost ion selective membrane 80 of the counter electrode assembly 14 allows passage of positively charged ions to the biological interface 18, while substantially blocking passage of ions having a negative charge or polarity. On the other hand, where the outermost ion selective membrane 38 of the active electrode assembly 12 allows passage of positively charged ions of the active agent 36, 40, 42 to the biological interface 18, the outermost ion selective membrane 80 of the counter electrode assembly 14 allows passage of negatively charged ions to the biological interface 18 while substantially blocking passage of ions with a positive charge or polarity.

The outer release liner 82 may generally be positioned overlying or covering an outer surface 84 of the outermost ion selective membrane 80. Note that the inner release liner 82 is shown in place in FIG. 1 and removed in FIG. 2. The outer release liner 82 may protect the outermost ion selective membrane 80 during storage, prior to application of an electromotive force or current. The outer release liner 82 may be a selectively releasable liner made of waterproof material, such as release liners commonly associated with pressure sensitive adhesives. In some embodiments, the outer release liner 82 may be coextensive with the outer release liner 46 of the active electrode assembly 12.

The voltage source 16 may take the form of one or more chemical battery cells, super- or ultra-capacitors, or fuel cells. The voltage source 16 may be selectively electrically coupled to the active and counter electrode assemblies 12,14 via a control circuit (not shown), which may include discrete and/or integrated circuit elements to control the voltage, current and/or power delivered to the electrode assemblies 12,14.

As suggested above, the active agent 36, 40, 42 may take the form of a cationic or an anionic drug or other therapeutic agent. Consequently, the terminals or poles 16a, 16b of the voltage source 16 may be reversed. Likewise, the selectivity of the outermost ion selective membranes 38, 80 and inner ion selective membranes 30, 74 may be reversed.

The iontophoresis device 10 may further comprise an inert molding material 86 adjacent exposed sides of the various other structures forming the active and counter electrode assemblies 12,14. The molding material 86 may advantageously provide environmental protection to the various structures of the active and counter electrode assemblies 12,14. Molding material 86 may form a slot or opening 88a on one of the exposed sides through which the tab 60 extends to allow for the removal of inner sealing liner 32 prior to use. Enveloping the active and counter electrode assemblies 12,14 is a housing material 90. The housing material 90 may also form a slot or opening 88b positioned aligned with the slot or opening 88a in molding material 86 through which the tab 60 extends to allow for the removal of inner sealing liner 32 prior to use of the iontophoresis device 10, as described below.

Immediately prior to use, the iontophoresis device 10 is prepared by withdrawing the inner sealing liner 32 and removing the outer release liners 46, 82. As described above, the inner sealing liner 32 may be withdrawn by pulling on tab 60. The outer release liners 46, 82 may be pulled off in a similar fashion to remove release liners from pressure sensitive labels and the like.

As best seen in FIG. 2, the active and counter electrode assemblies 12, 14 are positioned on the biological interface 18. Positioning on the biological interface may close the circuit, allowing electromotive force to be applied and/or current to flow from one pole 16a of the voltage source 16 to the other pole 16b, via the active electrode assembly, biological interface 18 and counter electrode assembly 14.

In the presence of the electromotive force and/or current, active agent 36 is transported toward the biological interface 18. Additional active agent 40 is released by the ion exchange groups or material 50 by the substitution of ions of the same charge or polarity (e.g., active agent 36), and transported toward the biological interface 18. While some of the active agent 36 may substitute for the additional active agent 40, some of the active agent 36 may be transferred through the outermost ion elective membrane 38 into the biological interface 18. Further active agent 42 carried by the outer surface 44 of the outermost ion elective membrane 38 is also transferred to the biological interface 18.

The above description of illustrated embodiments, including what is described in the Abstract, is not intended to be exhaustive or to limit the claims to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to other agent delivery systems and devices, not necessarily the exemplary iontophoresis active agent system and devices generally described above. For instance, some embodiments may include additional structure. For example, some embodiments may include a control circuit or subsystem to control a voltage, current or power applied to the active and counter electrode elements 24, 68. Also for example, some embodiments may include an interface layer interposed between the outermost ion selective membrane 38, 80 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240995, herein incorporated in their entirety by reference. Further examples of such hydrogels are disclosed in U.S. Patent Application Nos. 2004/166147; 2004/105834; and 2004/247655, herein incorporated in their entirety by reference. Product brand names of various hydrogels and hydrogel sheets include Corplex™ by Corium; Tegagel™ by 3M; PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

The various embodiments discussed above may advantageously employ various micro-structures, for example micro-needles. For example, a plurality of micro-needles may advantageously be formed on an outermost, biological interface contacting surface of the iontophoresis device.

Various microstructures, such as microneedles and microneedle arrays, and their manufacture and use have been described. Microneedles, either individually or in arrays, may be hollow; solid and permeable; solid and semi-permeable; or solid and non-permeable. Solid, non-permeable microneedles may further comprise grooves along their outer surfaces. Microneedles may be arranged in the form of arrays. Microneedles and microneedle arrays may be manufactured from a variety of materials, including silicon, silicon dioxide; molded plastic materials, including biodegradable or non-biodegradable polymers; ceramics, and metals. Microneedles, either individually or in arrays, may be used to dispense or sample fluids through the hollow apertures, through the solid permeable or semi-permeable materials, or via the external grooves. Microneedle devices are used, for example, to deliver a variety of compounds and compositions to the living body via a biological interface, such as skin or mucous membrane. In certain embodiments, the compounds and drugs may be delivered into or through the biological interface. For example, in delivering compounds or compositions via the skin, the length of the microneedle(s) or the depth of insertion may be used to control whether administration of a compound or composition is only into the epidermis, through the epidermis to the dermis, or subcutaneous. In certain embodiments, microneedle devices may be useful for delivery of high-molecular weight compounds and drugs, such as those comprising proteins, peptides and/or nucleic acids, and corresponding compositions thereof. In certain embodiments, for example wherein the fluid is an ionic solution, microneedle(s) or microneedle array(s) can provide electrical continuity between a voltage source and the tip of the microneedle(s). Microneedle(s) and microneedle array(s) may be used to deliver or sample compounds or compositions by iontophoretic methods, as disclosed herein. Such compounds or compositions may comprise, for example, high-molecular weight molecules or drugs, such as proteins, peptides and/or nucleic acids.

In certain embodiments, compounds or compositions can be delivered by an iontophoresis device comprising an active electrode assembly and a counter electrode assembly, electrically coupled to a voltage source to deliver an active agent to a biological interface. The active electrode assembly includes the following: a first electrode member connected to a positive electrode of the voltage source; a drug holding part having a drug solution that is in contact with the first electrode member and to which is applied a voltage via the first electrode member; a biological interface contact member, which is a microneedle array and is placed against the forward surface of the drug holding part; and a first cover or container that accommodates these members. The counter electrode assembly includes the following: a second electrode member connected to a negative electrode of the voltage source; a second electrolyte holding part that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a voltage source to deliver an active agent to a biological interface. The active electrode assembly includes the following: a first electrode member connected to a positive electrode of the voltage source; a first electrolyte holding part having an electrolyte that is in contact with the first electrode member and to which is applied a voltage via the first electrode member; a first anion-exchange membrane that is placed on the forward surface of the first electrolyte holding part; a drug holding part that is placed against the forward surface of the first anion-exchange membrane; a biological interface contacting member, which is a microneedle array and is placed against the forward surface of the drug holding part; and a first cover or container that accommodates these members. The counter electrode assembly includes the following: a second electrode member connected to a negative electrode of the voltage source; a second electrolyte holding part having an electrolyte that is in contact with the second electrode member and to which is applied a voltage via the second electrode member; a cation-exchange membrane that is placed on the forward surface of the second electrolyte holding part; a third electrolyte holding part that is placed against the forward surface of the cation-exchange membrane and holds an electrolyte to which a voltage is applied from the second electrode member via the second electrolyte holding part and the cation-exchange membrane; a second anion-exchange membrane placed against the forward surface of the third electrolyte holding part; and a second cover or container that accommodates these members.

Certain details of microneedle devices, their use and manufacture, are disclosed in U.S. Pat. Nos. 6,256,533; 6,312,612; 6,334,856; 6,379,324; 6,451,240; 6,471 ;903; 6,503,231; 6,511,463; 6,533,949; 6,565,532; 6,603,987; 6,611,707; 6,663,820; 6,767,341; 6,790,372; 6,815,360; 6,881,203; 6,908,453; 6,939,311; all of which are incorporated herein by reference in their entirety. Some or all of the above teaching therein may be applied to microneedle devices, their manufacture, and their use in iontophoretic applications.

The various embodiments described above can be combined to provide further embodiments. All of the U.S. patents, U.S. Patent Application publications, U.S. Patent Applications, foreign patents, foreign Patent Applications and non-patent publications referred to in this specification and/or listed in the Application Data Sheet are incorporated herein by reference, in their entirety, including but not limited to: Japanese Patent Application Serial No. H03-86002, filed Mar. 27, 1991, having Japanese Publication No. H04-297277, issued on Mar. 3, 2000 as Japanese Patent No. 3040517; Japanese Patent Application Serial No. 11-033076, filed Feb. 10, 1999, having Japanese Publication No. 2000-229128; Japanese Patent Application Serial No. 11-033765, filed Feb. 12, 1999, having Japanese Publication No. 2000-229129; Japanese Patent Application Serial No. 11-041415, filed Feb. 19, 1999, having Japanese Publication No. 2000-237326; Japanese Patent Application Serial No. 11-041416, filed Feb. 19, 1999, having Japanese Publication No. 2000-237327; Japanese Patent Application Serial No. 11-042752, filed Feb. 22, 1999, having Japanese Publication No. 2000-237328; Japanese Patent Application Serial No. 11-042753, filed Feb. 22, 1999, having Japanese Publication No. 2000-237329; Japanese Patent Application Serial No. 11-099008, filed Apr. 6, 1999, having Japanese Publication No. 2000-288098; Japanese Patent Application Serial No. 11-099009, filed Apr. 6, 1999, having Japanese Publication No. 2000-288097; PCT Patent Application No. WO 2002JP4696, filed May 15, 2002, having PCT Publication No. WO03037425; U.S. patent application Ser. No. 10/488970, filed Mar. 9, 2004; Japanese Patent Application 2004/317317, filed Oct. 29, 2004; U.S. provisional Patent Application Ser. No. 60/627,952, filed Nov. 16, 2004; Japanese Patent Application Serial No. 2004-347814, filed Nov. 30, 2004; Japanese Patent Application Serial No. 2004-357313, filed Dec. 9, 2004; Japanese Patent Application Serial No. 2005-027748, filed Feb. 3, 2005; Japanese Patent Application Serial No. 2005-081220, filed Mar. 22, 2005; and U.S. provisional Patent Application Ser. No. 60/721,843, filed Sep. 28, 2005.

Aspects of the various embodiments can be modified, if necessary, to employ systems, circuits and concepts of the various patents, applications and publications to provide yet further embodiments.

These and other changes can be made in light of the above detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

I/we claim:

1. An iontophoresis device, comprising:
an active electrode element operable to provide an electrical potential;
an outermost ion selective membrane having an outer surface; and
a first amount of active agent carried by at least a portion of the outer surface of the outermost ion selective membrane prior to use in the absence of an electromotive force or current.

2. The iontophoresis device of claim 1, further comprising:
a second amount of active agent cached within the outermost ion selective membrane.

3. The iontophoresis device of claim 2 wherein the first amount of active agent on the outer surface of the ion selective membrane has approximately the same composition as the second amount of active agent cached in the outermost ion selective membrane.

4. The iontophoresis device of claim 2 wherein the first amount of active agent on the outer surface of the ion selective membrane has a different composition than the second amount of active agent cached in the outermost ion selective membrane.

5. The iontophoresis device of claim 2, further comprising:
an inner active agent reservoir positioned between the active electrode element and the outermost ion selective membrane; and
a third amount of active agent stored in the inner active agent reservoir, the third amount of additional active agent having a polarity that is the same polarity as the second amount of active agent cached within the outermost ion selective membrane.

6. The iontophoresis device of claim 5 wherein the third amount of active agent stored in the inner active agent reservoir has approximately the same composition as the second amount of active agent cached in the outermost ion selective membrane.

7. The iontophoresis device of claim 5 wherein the third amount of active agent stored in the inner active agent reservoir has a different composition than that of the second amount of active agent cached in the outermost ion selective membrane.

8. The iontophoresis device of claim 5 wherein the outermost ion selective membrane is an ion exchange membrane having a plurality of pores, at least some of the pores including an ion exchange material to which the active agent binds.

9. The iontophoresis device of claim 8 wherein the third amount of active agent stored in the inner active agent reservoir at least partially replaces the second amount of active agent bound to the ion exchange groups of the outermost ion selective membrane when in use.

10. The iontophoresis device of claim 1 wherein the first amount of active agent forms a distinct layer on the outer surface of the outermost ion selective membrane.

11. The iontophoresis device of claim 10 wherein the distinctive layer is a gel layer.

12. The iontophoresis device of claim 2 wherein the first amount of active agent is deposited on the outer surface of the outermost ion selective membrane separately from loading the second amount of active agent in the outermost ion selective membrane.

13. The iontophoresis device of claim 2 wherein the outermost ion selective membrane substantially passes ions having a first polarity that matches a polarity of the second amount of active agent cached in the outermost ion selective membrane and substantially blocks passage of ions of a second polarity, opposite the first polarity.

14. The iontophoresis device of claim 13, further comprising:
an inner ion selective membrane selectively substantially passable by ions having the second polarity and substantially unpassable by ions having the first polarity; and
an electrolyte positioned between the active electrode element and the inner ion selective membrane.

15. The iontophoresis device of claim 14, further comprising:
an inner sealing liner withdrawably positioned between the electrolyte and any of the active agent.

16. The iontophoresis device of claim 15, further comprising:
a release agent on a side of the inner sealing liner that faces the inner ion selective membrane.

17. An iontophoresis device, comprising:
an active electrode element operable to provide an electrical potential;
an outermost ion selective membrane having an outer surface;
a first amount of active agent adhered to at least a portion of the outer surface of the outermost ion selective membrane; and
an outer release liner covering the first amount of active agent and at least a portion of the outer surface of the outermost selective membrane prior to use.

18. The iontophoresis device of claim 17, further comprising:
a second amount of active agent cached within the outermost ion selective membrane.

19. The iontophoresis device of claim 17, further comprising:
an electrolyte reservoir positioned between the active electrode element and the inner ion selective membrane.

20. The iontophoresis device of claim 17, further comprising:
a second amount of active agent cached within the outermost ion selective membrane;
an electrolyte reservoir positioned between the active electrode element and the inner ion selective membrane;
an inner active agent reservoir positioned between the electrolyte reservoir and the outermost ion selective membrane; and
a third amount of active agent stored in the inner active agent reservoir, the third amount of active agent having a polarity that is the same as a polarity as the second amount of active agent cached within the outermost ion selective membrane.

21. The iontophoresis device of claim 20, further comprising:
an inner sealing liner withdrawably positioned between the electrolyte reservoir and the inner active agent reservoir prior to use.

22. The iontophoresis device of claim 21 wherein the second amount of active agent cached in the outermost ion selective membrane, the first amount of active agent on the outer surface of the outermost ion selective membrane and the second amount of active agent stored in the inner active agent reservoir are of substantially identical composition.
